(19) United States
(12) Patent Application Publication
(10) Pub. No.: US 2025/0099545 A1
(43) Pub. Date: Mar. 27, 2025

(54) COMPOSITIONS, SYSTEMS, AND METHODS FOR TREATING CANCER USING TUMOR TREATING FIELDS WITH IMMUNE CHECKPOINT INHIBITORS AND MHC CLASS I ACTIVATORS

(71) Applicant: Novocure GmbH, Root D4 (CH)

(72) Inventors: Tal Kan, Haifa (IL); Tali Voloshin-Sela, Haifa (IL); Yiftah Barsheshet, Haifa (IL); Lilach Avigdor, Haifa (IL)

(73) Assignee: Novocure GmbH, Root D4 (CH)

(21) Appl. No.: 18/890,988

(22) Filed: Sep. 20, 2024

Related U.S. Application Data
(60) Provisional application No. 63/584,254, filed on Sep. 21, 2023.

(51) Int. Cl. A61K 38/21; A61K 45/06; A61N 1/40; A61P 35/00

(52) U.S. Cl. CPC A61K 38/217; A61K 45/06; A61N 1/40; A61P 35/00

(57) ABSTRACT

Compositions, systems, and methods for reducing viability of cancer cells and treating cancer, as well as preventing an increase of volume of a tumor present in a body of a living subject, are disclosed. The systems and methods involve application of an alternating field concurrently with administration of at least one composition that increases MHC Class I expression in the cancer cells.

### CROSS-REFERENCE TO RELATED APPLICATIONS

The subject application claims benefit under 35 USC § 119(e) of U.S. Provisional Application No. 63/584,254, filed Sep. 21, 2023. The entire contents of the above-referenced patent application(s) are hereby expressly incorporated herein by reference.

### STATEMENT REGARDING FEDERALLY SPONSORED RESEARCH

Not Applicable.

### BACKGROUND ART

Tumor Treating Fields (TTFields) are low intensity (e.g., 1-3 V/cm) alternating electric fields within the intermediate frequency range (such as, but not limited to, 100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the electrode arrays that make up each of these pairs are positioned on opposite sides of the body part that is being treated. More specifically, for the OPTUNE® system, one pair of electrodes is located to the left and right (LR) of the tumor, and the other pair of electrodes is located anterior and posterior (AP) to the tumor. TTFields are approved for the treatment of glioblastoma multiforme (GBM), and may be delivered, for example, via the OPTUNE® system (Novocure Limited, St. Helier, Jersey), which includes transducer arrays placed on the patient's shaved head.

Each transducer array used for the delivery of TTFields in the OPTUNE® device comprises a set of ceramic disk electrodes, which are coupled to the patient's skin (such as, but not limited to, the patient's shaved head for treatment of GBM) through a layer of conductive medical gel. The purpose of the medical gel is to deform to match the body's contours and to provide good electrical contact between the arrays and the skin; as such, the gel interface bridges the skin and reduces interference. The device is intended to be continuously worn by the patient for 2-4 days before removal for hygienic care and re-shaving (if necessary), followed by reapplication with a new set of arrays. As such, the medical gel remains in substantially continuous contact with an area of the patient's skin for a period of 2-4 days at a time. In addition, the arrays can be shifted a few centimeters in either direction to allow the skin to heal from one period of treatment to the next. Therefore, a portion of skin that was covered by electrodes/gel for a 2-4 day period could then be uncovered for 2-4 days when the replaced electrodes are shifted slightly; then the device may be reapplied to the original portion of skin for the next 2-4 day period.

TTFields have been studied clinically together with standard-of-care (SOC) immunotherapy or chemotherapy regimens for treatment of non-small cell lung cancer that has progressed on platinum-based therapy (Leal, et al. (2023) Lancet Oncol, 24(9):1002-1017) and preclinically together with immune checkpoint inhibitors (Voloshin, et al. (2020) Cancer Immunol Immunother, 69(7):1191-1204; Barsheshet, et al. (2022) Int J Mol Sci, 23(22):14073).

Downregulation of MHC Class I expression has been described in 40-90% of human tumors, often correlating with worse prognosis. Both adult and pediatric tumors can reduce MHC Class I-surface display using different regulatory mechanisms. The downregulation of MHC Class I allows tumors to avoid tumor-associated antigen presentation, and therewith T-cell-mediated cytotoxicity, by the downregulation of surface display of major histocompatibility complex (MHC) class I, a crucial factor in the initiation of an adaptive immune response (Cornel, et al. (2020) Cancers (Basel) 12(7):1760). Downregulation of MHC-I has been described as a mechanism of intrinsic and acquired resistance to immunotherapy in patients with cancer (Taylor, et al. (2022) Front Immunol, 13:844866). In addition, in pancreatic cancer, autophagy has been shown to be responsible for MHC Class I degradation, resulting in resistance to immunotherapy and inhibition of adaptive anti-tumor immune response (Yamamoto, et al. (2020) Nature, 581:100-105).

Therapeutically increasing MHC Class I expression has been shown to potentiate immune checkpoint blockade (Gu, et al. (2021) Cancer Discov, 11(6):1524-1541). In addition, the immunomodulatory properties of PI3K/AKT/mTOR and MAPK/MEK/ERK inhibition have been shown to augment the response to immune checkpoint blockade in melanoma and triple-negative breast cancer (Zhang, et al. (2022) Int J Mol Sci., 23(13):7353). Also, the United States Food and Drug Administration (FDA) approved the combination of PD-L1 inhibitor atezolizumab with BRAF inhibitor vemurafenib plus MEK inhibitor cobimetinib for the first-line treatment of advanced melanoma patients with BRAFV600 mutations in 2020. However, in patients with EGFR-mutant non-small cell lung cancer (NSCLC), the clinical benefits were not achieved when PD-L1 inhibitor durvalumab was combined with epidermal growth factor receptor-tyrosine kinase inhibitor (EGFR-TKI) osimertinib, and the combination only incurred more adverse events (Li, et al. (2023) Cancers, 15(10):2858).

TTFields have been shown to induce autophagy (Shteingauz, et al. (2018) Cell Death Dis., 9(11):1074; Davidi, et al. (2022) Cancers (Basel), 14(12):2959). In addition, autophagy has been shown to contribute to MHC class I degradation.

Inhibition of autophagy has been clinically tested using hydrochloroquine in breast cancer patients and using chloroquine in stage IV small cell lung cancer (SCLC) patients (US NLM Clinical Trials Identifiers NCT01292408 and NCT00969306, respectively); however, both of these trials were terminated due to poor results. Also, Interferon-gamma has been tested as a cancer therapy with no success (Zhang, et al. (2019) Cancer Immunol Res, 7(8):1237-1243). In addition, HDAC inhibitors are currently being tested in clinic, but have been shown to have severe side effects (Subramanian, et al. (2010) Pharmaceuticals (Basel), 3(9): 2751-2767; and Bondarev, et al. (2021) British J Clinical Pharmacol, 87(12):4577-4597).

Silginer et al. (Neuro-Oncology (2018) 20(6):vi133) investigated the interaction of TTFields with molecules involved in immune responses as well as drugs that may modulate immune cell activity and found increased NKG2D ligand expression and enhanced NK cell-based killing of glioma cells exposed to TTFields, while MHC Class I and Class II expression remained unaltered.

### BRIEF DESCRIPTION OF THE DRAWINGS

FIG. 1 graphically depicts an analysis of MHC Class I expression in human and mouse pancreatic cell lines (1A-AsPc1 human pancreatic cancer cell line and 1B-Panc02 mouse pancreatic cancer cell line) following TTFields exposures of 72 or 96 hours.

FIG. 2 graphically depicts transcriptomics analysis following TTFields treatment compared with control in glioblastoma (GBM) cell lines (GIN31, KNS 42, U87, GBM1, GBM, JHH-520, NCH466, SF188), lung adenocarcinoma cell lines (H-1650, HCC4006), ovarian cancer cell line (OVCAR3), gastric cancer cell lines (AGS, KATOIII), malignant pleural mesothelioma cell line (MPM), and pancreatic cancer cell lines (AsPC1 and BxPC3), to identify genes that exhibit a difference in expression following TTFields treatment.

FIG. 3 graphically depicts that MHC-I expression was reduced following TTFields application in Panc2 cells and increased with concomitant Chloroquine treatment. Panc02-luc mouse pancreatic cells (10,000 cells/well) were incubated for 72 h with the following conditions: Control untreated, 10 μM chloroquine (autophagy inhibitor), TTFields (1.75V/cm and 150 kHz), and TTFields plus 10 μM chloroquine.

FIG. 4 graphically depicts that MHC-I expression is reduced following TTFields application in A2780 cells and increased with concomitant Alpelisib treatment. A2780 human ovarian cancer cells (20,000 cells/well) were incubated for 144 h with the following conditions: Control untreated; 250 nM alpelisib (PI3K inhibitor) for 72 h—media containing the drug was removed, and fresh media without alpelisib was added for an additional 72 h incubation; TTFields (1.75V/cm and 200 kHz); TTFields plus 250 nM alpelisib for 72 h—media containing the drug was removed, and fresh media without alpelisib was added for an additional 72 h incubation under TTFields condition.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (e.g., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

The term “therapeutic composition” or “pharmaceutical composition” as used herein refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (e.g., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one condition, disease, and/or infection. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “concomitant therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously. In addition, it will be understood that one administration step (such as, but not limited to, administration of the TTFields) may occur over a longer period of time than the other administration step (i.e., oral administration or injection of a substance). In these instances of varying administration time periods, the term “simultaneously” will be understood to mean that the shorter administration step wholly overlaps with the longer administration step. However, the term “simultaneously” will include performing the shorter administration step at any point during the longer administration step (e.g., the beginning, middle, or end of the longer administration step, or any other time period therebetween), as well as performing the shorter administration step one or more times wholly within the time period of the longer administration step. Therefore, the term “simultaneously” does not require that the two administration steps be performed over the exact same length of time.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intratumoral, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

The term “target region,” as used herein, refers to a region containing all or a portion of the cancer, cancer cells, and/or tumor to be treated.

Turning now to the inventive concept(s), a concurrent therapy for cancer is disclosed herein. The concurrent therapy includes the use of alternating electric fields (e.g., TTFields) in addition with at least one immune checkpoint inhibitor compound and at least one compound that increases MHC Class I expression (wherein the two compounds may be in the same composition or separate compositions and may be administered simultaneously or wholly or partially sequentially). The combination of alternating electric fields (e.g., TTFields) with the immune checkpoint inhibitor and activator of MHC Class I expression provides a synergistic result in the treatment of cancer. Such methods may be carried out in vitro or in vivo.

The inventors have found that, surprisingly, concomitant treatment with TTFields and a drug that increases MHC class I expression can prevent MHC class I degradation and restore or even boost adaptive immunity. The inventors have further discovered that treatment of cancer with TTFields, a drug that increases MHC class I expression, and an immune checkpoint inhibitor can have a synergistic effect and can overcome tumor resistance to immune checkpoint inhibitors.

Certain non-limiting embodiments of the present disclosure are directed to a method of reducing viability of cancer cells. The method includes the steps of: (1) applying an alternating electric field to the cancer cells for a period of time; and (2) administering at least one composition to the cancer cells, wherein the at least one composition comprises at least one immune checkpoint inhibitor compound and at least one compound that increases MHC Class I expression.

Certain additional non-limiting embodiments of the present disclosure are directed to a method of treating cancer in a subject. The method includes the steps of: (1) applying an alternating electric field to a target region of the subject for a period of time; and (2) administering at least one composition to the subject, wherein the at least one composition comprises at least one immune checkpoint inhibitor compound and at least one compound that increases MHC Class I expression.

Certain additional non-limiting embodiments of the present disclosure are directed to a method of reducing a volume of a tumor present in a body of a living subject, wherein the tumor includes a plurality of cancer cells. The method includes the steps of: (1) applying an alternating electric field to a target region of the subject for a period of time; and (2) administering at least one composition to the subject, wherein the at least one composition comprises at least one immune checkpoint inhibitor compound and at least one compound that increases MHC Class I expression.

Certain additional non-limiting embodiments of the present disclosure are directed to a method of preventing an increase of volume of a tumor, wherein the tumor is present in a body of a living subject and includes a plurality of cancer cells. The method includes the steps of: (1) applying an alternating electric field to a target region of the subject for a period of time; and (2) administering at least one composition to the subject, wherein the at least one composition comprises at least one immune checkpoint inhibitor compound and at least one compound that increases MHC Class I expression.

Certain additional non-limiting embodiments of the present disclosure are directed to a method, comprising the steps of: (1) applying an alternating electric field to a target region of the subject for a period of time; and (2) administering at least one composition to the subject, wherein the at least one composition comprises at least one immune checkpoint inhibitor compound and at least one compound that increases MHC Class I expression.

Certain additional non-limiting embodiments of the present disclosure are directed to a method of reducing viability of cancer cells, the method comprising the steps of: (1) applying an alternating electric field to the cancer cells for a period of time; (2) administering at least one first composition to the cancer cells, wherein the at least one first composition comprises at least one immune checkpoint inhibitor; and (3) administering at least one second composition to the cancer cells, wherein the at least one second composition comprises at least one compound that increases MHC Class I expression in the cancer cells.

Certain additional non-limiting embodiments of the present disclosure are directed to a method of treating cancer in a subject, the method comprising the steps of: (1) applying an alternating electric field to a target region of the subject for a period of time; (2) administering at least one first composition to the subject, wherein the at least one first composition comprises at least one immune checkpoint inhibitor; and (3) administering at least one second composition to the subject, wherein the at least one second composition comprises at least one compound that increases MHC Class I expression in cancer cells in the subject.

Certain additional non-limiting embodiments of the present disclosure are directed to a method of reducing a volume of a tumor and/or preventing an increase of volume of the tumor, wherein the tumor is present in a body of a living subject and includes a plurality of cancer cells, the method comprising the steps of: (1) applying an alternating electric field to a target region of the subject for a period of time; (2) administering at least one first composition to the subject, wherein the at least one first composition comprises at least one immune checkpoint inhibitor; and (3) administering at least one second composition to the subject, wherein the at least one second composition comprises at least one compound that increases MHC Class I expression in cancer cells in the subject.

Certain additional non-limiting embodiments of the present disclosure are directed to a method, comprising the steps of: (1) applying an alternating electric field to a target region of the subject for a period of time; (2) administering at least one first composition to the subject, wherein the at least one first composition comprises at least one immune checkpoint inhibitor; and (3) administering at least one second composition to the subject, wherein the at least one second composition increases MHC Class I expression in cancer cells in the subject; and wherein steps (2) and (3) are performed simultaneously or wholly or partially sequentially. In a particular (but non-limiting) embodiment, administration of the alternating electric field increases the toxicity of the at least one first composition and/or the at least one second composition against cancer cells in the subject when compared to the administration of the at least one first composition and/or the at least one second composition to the subject in the absence of alternating electric field application.

Certain additional non-limiting embodiments of the present disclosure are directed to a composition that increases MHC Class I expression in cancer cells in the subject, for use in any of the methods disclosed herein above.

Certain additional non-limiting embodiments of the present disclosure are directed to a kit for reducing viability of cancer cells (or for performing any of the methods described herein above), the kit comprising a composition that increases MHC Class I expression in cancer cells in the subject; and a field generating device configured to apply an alternating electric field to the cancer cells for a period of time.

Certain additional non-limiting embodiments of the present disclosure are directed to a system comprising a first composition comprising at least one immune checkpoint inhibitor and a second composition that increases MHC Class I expression in cancer cells in the subject, for use in any of the methods disclosed herein above.

Certain additional non-limiting embodiments of the present disclosure are directed to a kit for reducing viability of cancer cells (or for performing any of the methods described herein above), the kit comprising: a first composition comprising at least one immune checkpoint inhibitor; a second composition that increases MHC Class I expression in cancer cells in the subject; and a field generating device configured to apply an alternating electric field to the cancer cells for a period of time.

Certain additional non-limiting embodiments of the present disclosure are directed to a system for reducing viability of cancer cells (or for performing any of the methods described herein above), the system comprising: a first composition comprising at least one immune checkpoint inhibitor; a second composition that increases MHC Class I expression in cancer cells in the subject; and a field generating device configured to apply an alternating electric field to the cancer cells for a period of time.

The steps of any of the methods of the present disclosure may be performed concomitantly or serially, and in particular, substantially simultaneously or wholly or partially sequentially. When the steps are performed wholly or partially sequentially, the at least one composition (or first and/or second compositions) may be administered together or separately, and each composition may be administered before or after application of the alternating electric field has begun.

The methods of the present disclosure may be utilized to treat any types of cancer cells/cancers/tumors that respond to treatment with alternating electric fields (e.g., TTFields), immune checkpoint inhibitors, and/or activators of MHC Class I expression. Non-limiting examples of cancer cells/cancers/tumors that can be treated in accordance with the present disclosure include hepatocellular carcinoma/carcinoma cells, glioblastoma/glioblastoma cells, pleural mesothelioma/mesothelioma cells, differentiated thyroid cancer/cancer cells, advanced renal cell carcinoma/carcinoma cells, ovarian cancer/cancer cells, pancreatic cancer/cancer cells, lung cancer/cancer cells, breast cancer/cancer cells, and the like, as well as any combination thereof.

In a particular (but non-limiting) embodiment, the cancer may be a solid tumor.

Any type of conductive or non-conductive electrode(s) and/or transducer array(s) that can be utilized for generating an alternating electric field that are known in the art or otherwise contemplated herein may be utilized for generation of the alternating electric field in accordance with the methods of the present disclosure. Non-limiting examples of electrodes and transducer arrays that can be utilized for generating an alternating electric field in accordance with the present disclosure include those that function as part of an alternating electric field-generating system (e.g., a TTFields system) as described, for example but not by way of limitation, in U.S. Pat. Nos. 7,016,725; 7,089,054; 7,333,852; 7,565,205; 8,244,345; 8,715,203; 8,764,675; 10,188,851; and 10,441,776; and in US Patent Application Publication Nos. US 2018/0160933; US 2019/0117956; US 2019/0307781; and US 2019/0308016.

The alternating electric field may be generated at any frequency in accordance with the present disclosure. For example (but not by way of limitation), the alternating electric field may have a frequency of about 50 kHz, about 60 kHz, about 70 kHz, about 75 kHz, about 80 kHz, about 90 kHz, about 100 kHz, about 105 kHz, about 110 kHz, about 115 kHz, about 120 kHz, about 125 kHz, about 130 kHz, about 135 kHz, about 140 kHz, about 145 kHz, about 150 kHz, about 155 kHz, about 160 kHz, about 165 kHz, about 170 kHz, about 175 kHz, about 180 kHz, about 185 kHz, about 190 kHz, about 195 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 550 kHz, about 600 kHz, about 650 kHz, about 700 kHz, about 750 kHz, about 800 kHz, about 850 kHz, about 900 kHz, about 950 kHz, about 1 MHz, about 2 MHz, about 3 MHz, about 4 MHz, about 5 MHz, about 6 MHz, about 7 MHz, about 8 MHz, about 9 MHz, about 10 MHz, and the like, as well as a range formed from any of the above values (e.g., a range of from about 50 kHz to about 10 MHz, a range of from about 50 kHz to about 1 MHz, a range of from about 50 kHz to about 500 kHz, a range of from about 100 kHz to about 500 kHz, a range of from about 150 kHz to about 300 kHz, etc.), and a range that combines two integers that fall between two of the above-referenced values (e.g., a range of from about 122 kHz to about 313 kHz, a range of from about 78 kHz to about 298 kHz, etc.).

In certain particular (but non-limiting) embodiments, the alternating electric field may be imposed at two or more different frequencies. When two or more frequencies are present, each frequency is selected from any of the above-referenced values, or a range formed from any of the above-referenced values, or a range that combines two integers that fall between two of the above-referenced values.

In certain particular (but non-limiting) embodiments, the following frequencies may be utilized for specific cancers: GBM, about 200 kHz; NSCLC, about 150 kHz; breast cancer, about 200 kHz; pancreatic cancer, about 150 kHz; brain metastases from NSCLC, about 150 kHz; hepatic cancer, about 150 kHz; and the like.

The alternating electric field may have any field strength in the target region/subject/cancer cells, so long as the alternating electric field is capable of functioning in accordance with the present disclosure. For example (but not by way of limitation), the alternating electric field may have a field strength in the target region/subject/cancer cells of at least about 1 V/cm, about 1.5 V/cm, about 2 V/cm, about 2.1 V/cm, about 2.2 V/cm, about 2.3 V/cm, about 2.4 V/cm, about 2.5 V/cm, about 2.6 V/cm, about 2.7 V/cm, about 2.8 V/cm, about 2.9 V/cm, about 3 V/cm, about 3.5 V/cm, about 4 V/cm, about 4.5 V/cm, about 5 V/cm, about 5.5 V/cm, about 6 V/cm, about 6.5 V/cm, about 7 V/cm, about 7.5 V/cm, about 8 V/cm, about 9 V/cm, about 9.5 V/cm, about 10 V/cm, about 10.5 V/cm, about 11 V/cm, about 11.5 V/cm, about 12 V/cm, about 12.5 V/cm, about 13 V/cm, about 13.5 V/cm, about 14 V/cm, about 14.5 V/cm, about 15 V/cm, about 15.5 V/cm, about 16 V/cm, about 16.5 V/cm, about 17 V/cm, about 17.5 V/cm, about 18 V/cm, about 18.5 V/cm, about 19 V/cm, about 19.5 V/cm, about 20 V/cm, and the like, as well as a range formed from any of the above values (e.g., a range of from about 1 V/cm to about 20 V/cm, a range of from about 1 V/cm to about 10 V/cm, a range of from about 1 V/cm to about 4 V/cm, etc.), and a range that combines two integers that fall between two of the above-referenced values (e.g., a range of from about 1.1 V/cm to about 18.6 V/cm, a range of from about 1.2 V/cm to about 9.8 V/cm, a range of from about 1.3 V/cm to about 4.7 V/cm, etc.). Generally, it is desired to utilize the highest field strength possible without causing overheating, with field intensity typically being capped by temperature measurements.

In some instances, the electric field in at least a portion of the target region/subject/cancer cells is induced by an applied voltage that is determined by computer simulation of the target region/subject/cancer cells. In some instances, the electric field in at least a portion of the target region/subject/cancer cells is induced by an applied voltage of at least 50 V RMS (root mean squared) or at least 50 V p2p (peak-to-peak), and optionally, the applied voltage is at least 100 V RMS or at least 100 V p2p. In some embodiments, an applied voltage of at least 50 V induces an electric field with a field strength of at least 1 V/cm (e.g., at least 5 V/cm) in at least a portion of the target region/subject/cancer cells.

The alternating electric field may be applied in a single direction between a pair of arrays or may be alternating in two or more directions/channels between two or more pairs of arrays (e.g., front-back and left-right). For example, certain TTFields devices (such as, but not limited to, the OPTUNE® system (Novocure Limited, St. Helier, Jersey)) operate in two directions in order to increase the chances that a dividing cell will be aligned with the electric field such that the electric field can have the desired anti-mitotic effect. However, it will be understood that the scope of the present disclosure also includes the application of the alternating electric field in a single direction. The term “alternating electric field” as used herein will be understood to include application in a single direction/channel as well as in two or more directions/channels; in addition, the term “alternating electric field” as used herein will be understood to include both application of a single alternating electric field as well as application of a plurality of alternating electric fields in succession for a duration of time.

The alternating electric field may be applied for any continuous or cumulative period of time sufficient to achieve a reduction in viability of cancer cells and/or a reduction in tumor volume (and/or a prevention of increase in tumor volume). The period of time that the alternating electric field is applied includes both a continuous period of time as well as a cumulative period of time. That is, the period of time that the alternating electric field is applied includes a single session (i.e., continuous application) as well as multiple sessions with minor breaks in between sessions (i.e., consecutive application for a cumulative period). For example, a subject is allowed to take breaks during treatment with an alternating electric field device and is only expected to have the device positioned on the body and operational for at least about 50%, at least about 60%, at least about 70%, or at least about 80% of the total treatment period (e.g., over a course of one day, one week, two weeks, one month, two months, three months, four months, five months, etc.).

For example, but not by way of limitation, the alternating electric field may be applied for a continuous or cumulative period of time of at least about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, about 8 days, about 9 days, about 10 days, about 11 days, about 12 days, about 13 days, about 14 days, about 21 days, about 1 month, about 2 months, about 3 months, about 4 months, about 5 months, about 6 months, and the like, as well as a range formed from any of the above values (e.g., a range of from about 1 hour to about 6 months, a range of from about 1 hour to about 7 days, a range of from about 24 hours to about 72 hours, etc.), and a range that combines two integers that fall between two of the above-referenced values (e.g., a range of from about 14 hours to about 68 hours, etc.).

In a particular (but non-limiting) embodiment, the period of time that the alternating electric field is applied is at least about 24 cumulative hours within 48 consecutive hours. In another particular (but non-limiting) embodiment, the period of time that the alternating electric field is applied is at least about 24 hours, with the device positioned on the body and operational for at least about 80% of that period.

The total period of time that the alternating electric field is applied may be achieved in a continuous or intermittent manner. That is, when the alternating electric field is applied for a shorter period of time (such as, but not limited to, less than about 12 or 24 hours), the alternating electric field may be continuously applied over that period of time. However, when the alternating electric field is applied for a longer period of time (such as, but not limited to, a period of about 24 hours or greater), the treatment period may include one or more breaks during the application cycle that separate two or more application sections, whereby the application sections and breaks combine to form the total application period. When breaks are present, the alternating electric field is applied for at least about 50%, about 60%, about 70%, about 80%, or about 90% or more of the treatment time, so that the breaks typically constitute only about 10%, about 20%, about 30%, about 40%, about 50%, or less of the treatment time. In a particular (but non-limiting) embodiment, when breaks are present, the breaks should typically constitute about 20% or less of the treatment time, so that the alternating electric field is applied for at least about 80% or more of the treatment time. For example, but not by way of limitation, the alternating electric field should be applied for at least about 19 hours of each 24-hour period. In addition, the longer that the alternating electric field is applied, the higher the efficacy will be.

Suitable types of immune checkpoint inhibitors that may be utilized in accordance with the present disclosure include, but are not limited to, PD-1 inhibitors, PDL-1 inhibitors, CTLA-4 inhibitors, TIM3 inhibitors, TIGIT inhibitors, LAG3 inhibitors, agonist antibodies targeting receptors such as (but not limited to) 4-1BB and OX40, and the like. Particular (but non-limiting) examples of anti-PD-1 IgG4 that may be utilized in accordance with the present disclosure include nivolumab, pembrolizumab, cemiplimab, spartalizumab, tislelizumab, and the like. Particular (but non-limiting) examples of anti-PD-L1 IgG1 that may be utilized in accordance with the present disclosure include atezolizumab, durvalumab, avelumab, BGB-A333, and the like. Particular (but non-limiting) examples of anti-CTLA4 compounds that may be utilized in accordance with the present disclosure include ipilimumab, tremelimumab, and the like. A particular (but non-limiting) example of an anti-TIM3 IgG4 that may be utilized in accordance with the present disclosure is sabatolimab and the like. Particular (but non-limiting) examples of anti-TIGIT IgG1 that may be utilized in accordance with the present disclosure include vibostolimab, tiragolumab, and the like. Particular (but non-limiting) examples of anti-LAG3 compounds that may be utilized in accordance with the present disclosure include relatlimab, ieramilimab, and the like, as well as eftilagimod alpha (soluble LAG-3 protein).

Any compositions that function as MHC Class I activators to increase MHC Class I expression that are known in the art or are otherwise contemplated herein may be utilized in accordance with the present disclosure, so long as the compositions are capable of functioning as described herein. Suitable MHC Class I activators that may be utilized in accordance with the present disclosure include PI3K inhibitors, autophagy inhibitors, HDAC inhibitors, TRAF3 inhibitors, topotecan, interferon-gamma, BRAF inhibitors, MEK inhibitors, vemurafenib, cobimetinib, and the like, as well as any combinations thereof.

Non-limiting examples of PI3K inhibitors that may be utilized in accordance with the present disclosure include alpelisib, dactolisib, PI-103, pictilisib, buparlisib, copanlisib, duvelisib, taselisib, pilaralisib, voxtalisib, nemiralisib, umbralisib, GDC-0326, SF2523, serabelisib, eganelisib, tenalisib, seletalisib, leniolisib, acalisib, parsaclisib, ME401, inavolisib, HS-173, PI-3065, GNE-317,CZC24832, GSK2636771, PF-4989216, AZD8186, AMG319, A66, AS-252424, AS-604850, CAY1505, NU75-441, CH5132799, GDC-0941, TG100-115, TG100713, CH5132799, PX-866, LY294002, XL147, ZSTK474, BKM-120, BAY80-6946, AZD8835, WX-037, KA2237, CAL-120, INCB050465, INK-1117, TGR-1202, RP6530, GDC-0032, BYL719, IPI-145, CAL-101, PIK-90,PIK-294, IC-87114, AS-605240, AZD6482, AMG511, ADZ6482, MLN1117, 3-hydroxyanthranilic acid, hispidulin, pectolinarin, cinobufagin, and the like.

Non-limiting examples of autophagy inhibitors that may be utilized in accordance with the present disclosure include chloroquine, hydroxychloroquine, cycloheximide, bafilomycin A1, Lys05, 3-methyladenine (3-MA), LY294002, Wortmaninn, leupeptin, E64d, pepstatin A, Spautin 1, MRT 67307, MRT 68921, and the like.

Non-limiting examples of HDAC inhibitors that may be utilized in accordance with the present disclosure include apicidin, entinostat, MS-275, sodium butyrate (NaB, butanoic acid sodium salt), suberoyanilide hydroxamic acid (SAHA; vorinostat), trichostatin A (TSA), valproic acid, panobinostat, belinostat, abexinostat, dacinostat, quisinostat, mocetinostat, valproic acid sodium salt (NSC 93819, sodium valproate), CUDC-101, droxinostat, MC1568, pracinostat, divalproex sodium, givinostat, M344, romidepsin, tacedinaline, Scriptaid (GCK 1026), resminostat (RAS2410), RGFP966, RG2833, TMP269, Santacruzamate A (CAY10683), tasquinimod (ABR-215050), LMK-235, CAY10603, domatinostat, BG45, BRD72954, TMP196, tucidinostat, AR-42, citarinostat (ACY-241), GSK3117391, biphenyl-4-sulfonyl chloride, UF010, suberohydroxamic acid, NKL22, TC-H 106, SR-4370, SIS17, BRD3308, CXD101, and the like.

One non-limiting example of a BRAF inhibitors that may be utilized in accordance with the present disclosure is vemurafenib.

One non-limiting example of an MEK inhibitors that may be utilized in accordance with the present disclosure is cobimetinib.

The composition(s) of the present disclosure (i.e., containing the immune checkpoint inhibitor(s) and/or the MHC Class I activator(s)) may be provided with any formulation known in the art or otherwise contemplated herein. In certain particular (but non-limiting) embodiments, the compositions contain one or more pharmaceutically acceptable carriers (and as such, the composition may also be referred to as a “pharmaceutical composition”). Non-limiting examples of suitable pharmaceutically acceptable carriers include water; saline; dextrose solutions; fructose or mannitol; calcium carbonate; cellulose; ethanol; oils of animal, vegetative, or synthetic origin; carbohydrates, such as glucose, sucrose, or dextrans; antioxidants, such as ascorbic acid or glutathione; chelating agents; low molecular weight proteins; detergents; liposomal carriers; nanocarriers; scaffolds that allowed delayed drug release (such as, but not limited to, hydrogels); buffered solutions, such as sodium chloride, saline, phosphate-buffered saline, and/or other substances which are physiologically acceptable and/or safe for use; diluents; excipients such as polyethylene glycol (PEG); or any combination thereof. Suitable pharmaceutically acceptable carriers for pharmaceutical formulations are described, for example, in Remington: The Science and Practice of Pharmacy, 23rd ed. (2020).

In certain particular (but non-limiting) embodiments, the composition(s) of the present disclosure (i.e., containing the immune checkpoint inhibitor(s) and/or the MHC Class I activator(s)) may further contain one or more additional active agents. Various active agents that can be utilized concurrently with alternating electric fields, immune checkpoint inhibitors, and/or MHC Class I expression activators are known in the art, and certain combination therapies are approved by the FDA or currently in clinical trials testing.

In addition, any of the compositions of the present disclosure may contain other agents that allow for administration of the compositions via a particular administration route. For example, but not by way of limitation, the compositions may be formulated for administration by oral, topical, transdermal, parenteral, subcutaneous, intranasal, mucosal, intramuscular, intraperitoneal, intravitreal, intratumoral, and/or intravenous routes. Based on the route of administration, the compositions may also contain one or more additional components in addition to the active agent(s) (e.g., immune checkpoint inhibitor(s) and/or MHC Class I expression activator(s) and/or additional therapeutic agent(s)). Examples of additional secondary compounds that may be present include, but are not limited to, fillers, salts, buffers, preservatives, stabilizers, solubilizers, wetting agents, emulsifying agents, dispersing agents, gels, adhesives, and other materials well known in the art.

In a particular (but non-limiting) embodiment, any of the compositions of the present disclosure (i.e., containing the immune checkpoint inhibitor(s) and/or the MHC Class I activator(s)) is administered via injection or implantation into the subject. For example (but not by way of limitation), in some instances, it may be desired that the composition(s) be administered on a local/regional level to ensure targeting of the composition(s) to a specific location in the body of the subject and inhibit non-specific interactions in other parts of the body; in other instances, a more systemic administration may be desired.

Any of the compositions of the present disclosure (i.e., containing the immune checkpoint inhibitor(s) and/or the MHC Class I activator(s)) may be administered before or after application of the alternating electric field has begun. In certain particular (but non-limiting) embodiments, at least one composition may be administered before the application of the alternating electric field has begun. In other particular (but non-limiting) embodiments, at least one composition may be administered after the application of the alternating electric field has begun. In particular (but not by way of limitation), the composition(s) may be administered during application of the alternating electric field (e.g., before the period of time that the alternating electric field is applied has elapsed) and/or after application of the alternating electric field has elapsed. In certain particular (but non-limiting) embodiments, at least one composition may be administered before application of the alternating electric field has begun, and at least one composition may be administered after application of the alternating electric field has begun (and before or after application of the alternating electric field has elapsed).

For example (but not by way of limitation), any of the compositions of the present disclosure (i.e., containing the immune checkpoint inhibitor(s) and/or the MHC Class I activator(s)) may be administered before application of the alternating electric field has commenced by a period of at least about 1 minute, about 5 minutes, about 10 minutes, about 15 minutes, about 30 minutes, about 45 minutes, about 1 hour, about 1.5 hours, about 2 hours, about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the at least one composition that increases MHC Class I expression is administered at least about 24 hours before application of the alternating electric field has begun.

In other non-limiting examples, any of the compositions of the present disclosure (i.e., containing the immune checkpoint inhibitor(s) and/or the MHC Class I activator(s)) may be administered after application of the alternating electric field has commenced by a period of at least about 1 minute, about 5 minutes, about 10 minutes, about 15 minutes, about 30 minutes, about 45 minutes, about 1 hour, about 1.5 hours, about 2 hours, about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the at least one composition that increases MHC Class I expression is administered at least about 24 hours after application of the alternating electric field has begun.

In yet other non-limiting examples, any of the compositions of the present disclosure (i.e., containing the immune checkpoint inhibitor(s) and/or the MHC Class I activator(s)) may be administered after the period of time that the alternating electric field is applied has elapsed, wherein the one or more composition(s) is administered within about 1 minute, about 5 minutes, about 10 minutes, about 15 minutes, about 30 minutes, about 45 minutes, about 1 hour, about 1.5 hours, about 2 hours, about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 5 days, about 6 days, about 7 days, and the like, of when the period of time elapsed.

In a particular (but non-limiting) embodiment, one or more of the compositions of the present disclosure (i.e., containing the immune checkpoint inhibitor(s) and/or the MHC Class I activator(s)) is administered within about 96 hours of when the period of time elapsed.

The compositions of the present disclosure (i.e., containing the immune checkpoint inhibitor(s) and/or the MHC Class I activator(s)) may be administered to the cancer cells/subject at any concentration that provides a therapeutically effective concentration of the active agent(s). In certain non-limiting embodiments, the application of the alternating electric field reduces the amount of the active agent required to be therapeutically effective when compared to a normal therapeutically effective amount of active agent administered in the absence of an alternating electric field. For example, but not by way of limitation, the therapeutically effective concentration of the composition may be reduced by at least about 25%, about 30%, about 35%, about 40%, about 45%, about 50%, about 55%, about 60%, about 65%, about 70%, about 75% or more with respect to a dosage of the composition known to be therapeutically effective in the absence of application of an alternating electric field. In a particular (but non-limiting) embodiment, the therapeutically effective concentration of the composition is reduced by at least about 50% when compared to a dosage of the composition known to be therapeutically effective in the absence of an alternating electric field.

The therapeutically effective concentration of each active agent (immune checkpoint inhibitor or MHC Class I activator) utilized in accordance with the present disclosure may be, for example (but not by way of limitation), about 1 nM, about 10 nM, about 20 nM, about 30 nM, about 40 nM, about 50 nM, about 60 nM, about 70 nM, about 80 nM, about 90 nM, about 100 nM, about 125 nM, about 150 nM, about 175 nM, about 200 nM, about 250 nM, about 300 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 25 mM, about 30 mM, about 35 mM, about 40 mM, about 45 mM, about 50 mM, and the like, as well as a range formed from any of the above values (e.g., a range of from about 12.5 nM to about 100 nM, a range of from about 1 mM to about 20 mM, etc.), and a range that combines two integers that fall between two of the above-referenced values (e.g., a range of from about 17 nM to about 83 nM, etc.).

In a particular (but non-limiting) embodiment, the therapeutically effective concentration of each active agent is from about 10 nM to about 100 nM.

In particular (but non-limiting) embodiments, the therapeutically effective concentration of each active agent utilized in accordance with the present disclosure may be, for example (but not by way of limitation), about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, about 40 mg/kg, about 41 mg/kg, about 42 mg/kg, about 43 mg/kg, about 44 mg/kg, about 45 mg/kg, about 46 mg/kg, about 47 mg/kg, about 48 mg/kg, about 49 mg/kg, about 50 mg/kg, about 51 mg/kg, about 52 mg/kg, about 53 mg/kg, about 54 mg/kg, about 55 mg/kg, about 56 mg/kg, about 57 mg/kg, about 58 mg/kg, about 59 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, and the like, as well as a range formed from any of the above values (e.g., a range of from about 10 mg/kg to about 50 mg/kg, a range of from about 1 mg/kg to about 40 mg/kg, a range of from about 1 mg/kg to about 30 mg/kg, a range of from about 1 mg/kg to about 25 mg/kg, a range of from about 1 mg/kg to about 20 mg/kg, a range of from about 1 mg/kg to about 10 mg/kg, etc.).

In certain particular (but non-limiting) embodiments, the method includes one or more additional steps. For example (but not by way of limitation), the method may further include the step of: discontinuing the application of the alternating electric field (such as, but not limited to, to allow the cells/tissue to recover). In addition, any of the steps may be repeated one or more times.

In certain particular (but non-limiting) embodiments, any of the compositions of the present disclosure (i.e., containing the immune checkpoint inhibitor(s) and/or the MHC Class I activator(s)) may be administered by any dosage regimen known in the art. For example, but not by way of limitation, each composition may be administered in a single dosage or multiple dosages over a defined treatment period. For example (but not by way of limitation), a therapeutically effective concentration of one or more compositions may be administered about once every 4 hours, about once every 8 hours, about once every 12 hours, about once every day, about once every other day, about once every three days, about once a week, about twice a week, about three times a week, about once every two weeks, about once every three weeks, about once a month, and the like, as well as a range formed from any of the above values (a range of about once every 4 to 8 hours, a range of from about once a week to about once a month, etc.).

In addition, when multiple compositions are administered (i.e., the immune checkpoint inhibitor(s) and MHC Class I activator(s) are present in different compositions), the two or more compositions may be administered via the same route (e.g., both administered intravenously), or the two or more compositions may be administered by different routes (e.g., one composition orally administered and another composition intravenously administered).

In certain particular (but non-limiting) embodiments, the method involves concurrent therapy with yet additional compositions. As such, the method may include an additional step of administering at least one additional composition (other than the composition(s) containing the immune checkpoint inhibitor(s) and/or MHC Class | expression activator(s)) to the cancer cells/subject.

When present, administration of the at least one additional composition may be performed substantially simultaneously or wholly or partially sequentially with the administration of any of the composition(s) containing the immune checkpoint inhibitor(s) and/or MHC Class I expression activator(s), whereby the separate compositions are administered simultaneously or wholly or partially sequentially. Also, when the method includes administration of the additional composition, the optional administration step may be performed before or after the application of the alternating electric field has begun, during application of the alternating electric field, and/or after application of the alternating electric field has elapsed, in the same manner(s) and time frame(s) as described above for the other composition(s).

In certain particular (but non-limiting) embodiments, the method may further comprise the step of administering at least one additional therapy to the cells/subject. Any therapies known in the art or otherwise contemplated herein for use with alternating electric fields (e.g., TTFields), immune checkpoint inhibitor(s), and/or MHC Class I expression activator(s) may be utilized in accordance with the methods of the present disclosure. Non-limiting examples of additional therapies that may be utilized include radiation therapy (such as, but not limited to, ionizing radiation therapy), photodynamic therapy, transarterial chemoembolization (TACE), or combinations thereof.

Any of the method steps may be repeated one or more times. Each of the steps can be repeated as many times as necessary. When the step of applying the alternating electric field is repeated, the transducer arrays may be placed in slightly different positions on the subject than their original placement; relocation of the arrays in this manner may further aid in treatment of the tumor/cancer. In addition, any of the steps of administering any of the compositions/additional therapies may be repeated various times and at various intervals to follow any known and/or generally accepted dosage/treatment regimen for the composition(s)/therapy(ies).

While the methods described herein above are related to use of the combination of alternating electric fields (e.g., TTFields) with immune checkpoint inhibitor(s) and/or MHC Class I expression activator(s) in cancer treatment, it will be understood that the scope of the present disclosure is not limited to use in cancer treatment. Rather, the present disclosure encompasses treatment of any other related diseases, infections, or conditions for which immune checkpoint inhibitor(s) and/or MHC Class I expression activator(s) treatment and/or alternating electric field treatment is beneficial.

Certain non-limiting embodiments of the present disclosure are directed to any of the compositions disclosed or otherwise contemplated herein that increases MHC Class I expression in cancer cells in the subject, and wherein the composition is for use in any of the methods disclosed or otherwise contemplated herein.

Certain non-limiting embodiments of the present disclosure are related to kits that include any of the components of the alternating electric field (e.g., TTFields) generating systems disclosed or otherwise contemplated herein (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with at least one of any of the compositions (containing immune checkpoint inhibitor(s) and/or MHC Class I expression activator(s)) disclosed or otherwise contemplated herein. The kits may optionally further include one or more of any of the optional compositions disclosed or otherwise contemplated herein (such as, but not limited to, one or more optional compositions containing at least one additional active agent). The kits may optionally further include one or more devices (or one or more components of devices) utilized in one or more additional therapy steps.

In a particular (but non-limiting) embodiment, the kit may further include instructions for performing any of the methods disclosed or otherwise contemplated herein. For example (but not by way of limitation), the kit may include instructions for applying one or more components of the alternating electric field (e.g., TTFields) generating device to the skin of the patient, instructions for applying the alternating electric field to the patient, instructions for formulating one or more of the compositions, instructions for when and how to administer the one or more compositions, and/or instructions for when to activate and turn off the alternating electric field in relation to the administration of the composition(s) and/or optional therapy steps.

In addition to the components described in detail herein above, the kits may further contain other component(s)/reagent(s) for performing any of the particular methods described or otherwise contemplated herein. For example (but not by way of limitation), the kits may additionally include: (i) components for preparing the skin prior to disposal of the hydrogel compositions and/or transducer arrays thereon (e.g., a razor, a cleansing composition or wipe/towel, etc.); (ii) components for removal of the gel/transducer array(s); (iii) components for cleansing of the skin after removal of the gel/transducer array(s); and/or (iv) other components utilized with the system (e.g., conductive material, nonconductive material, a soothing gel or cream, a bandage, etc.). The nature of these additional component(s)/reagent(s) will depend upon the particular treatment format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In certain non-limiting embodiments, the kit further comprises a sealed packaging in which the components are disposed. In certain particular (but non-limiting) embodiments, the sealed packaging is substantially impermeable to air and/or substantially impermeable to light.

In addition, the kit can further include a set of written instructions explaining how to use one or more components of the kit. A kit of this nature can be used in any of the methods described or otherwise contemplated herein.

In certain non-limiting embodiments, the kit has a shelf life of at least about six months, such as (but not limited to), at least about nine months, or at least about 12 months.

Certain non-limiting embodiments of the present disclosure are related to systems that include any of the components of the alternating electric field generating systems disclosed or otherwise contemplated herein (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with at least one of any of the compositions (containing immune checkpoint inhibitor(s) and/or MHC Class I expression activator(s)) disclosed or otherwise contemplated herein. The systems may optionally further include one or more of any of the optional compositions disclosed or otherwise contemplated herein. The systems may optionally further include one or more devices (or one or more components of devices) utilized in one or more additional therapy steps.

### EXAMPLES

Examples are provided herein below. However, the present disclosure is to be understood to not be limited in its application to the specific experimentation, results, and laboratory procedures disclosed herein after. Rather, the Examples are simply provided as one of various embodiments and is meant to be exemplary, not exhaustive.

### Example 1

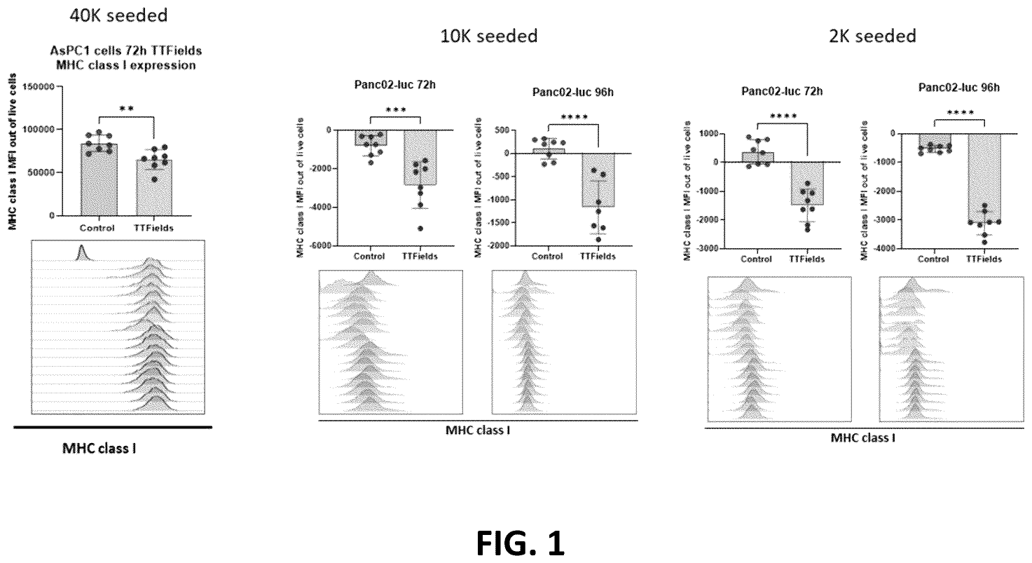

In this Example, the effects of TTFields on MHC class I expression in cancer cell lines was examined. In contrast to the prior art of Silginer et al. (2018) referenced in the Background section above, which found that MHC Class I and Class II expression remained unaltered following exposure to TTFields, this Example found that MHC class I expression was actually reduced in human and mouse pancreatic cancer cell lines following exposure to TTFields. As shown in FIG. 1, MHC class I expression was reduced in the 1A-AsPc1 human pancreatic cancer cell line following a 72-hour exposure to TTFields (left panel), and MHC class I expression was reduced in the 1B-Panc02 mouse pancreatic cancer cell line following 72- or 96-hour exposures to TTFields (middle and right panels).

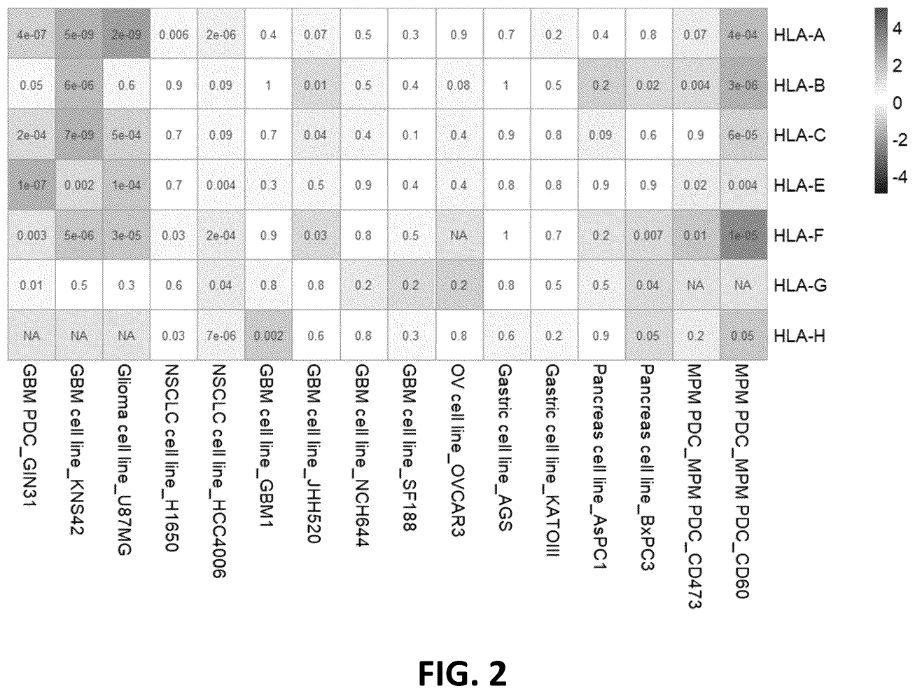

Furthermore, a transcriptomics analysis was conducted to identify class I HLA genes that exhibit differences in expression following TTFields treatment of various cancer cell lines compared with control. As shown in FIG. 2, there is a statistically significant reduction in various class I HLAs tested (including HLA-A, HLA-B, HLA-C, HLA-E, HLA-F, HLA-G, and HLA-H) following TTFields in glioblastoma (GBM) cell lines, malignant pleural mesothelioma cell line (MPM), and pancreatic cancer cell lines AsPC1 and BxPC3. Therefore, this data demonstrates that TTFields decrease MHC class I expression in various cancers.

While not wishing to be bound by any theory, it has previously been shown that TTFields induce ER stress (Silginer et al. (2017) Cell Death Dis, 8:e2753). In addition, it has also been shown previously that ER stress downregulates class I HLA (Ulianich et al. (2011) Biochemica et. Biophysica Acta, 1812:431-438). Therefore, the induction of ER stress by TTFields exposure could be a possible mechanism for downregulation of class I HLA following TTFields exposure.

### Example 2

In this Example, the combined effects of concurrent therapy with TTFields and an MHC Class I expression activator (such as, but not limited to, chloroquine and alpelisib) were examined.

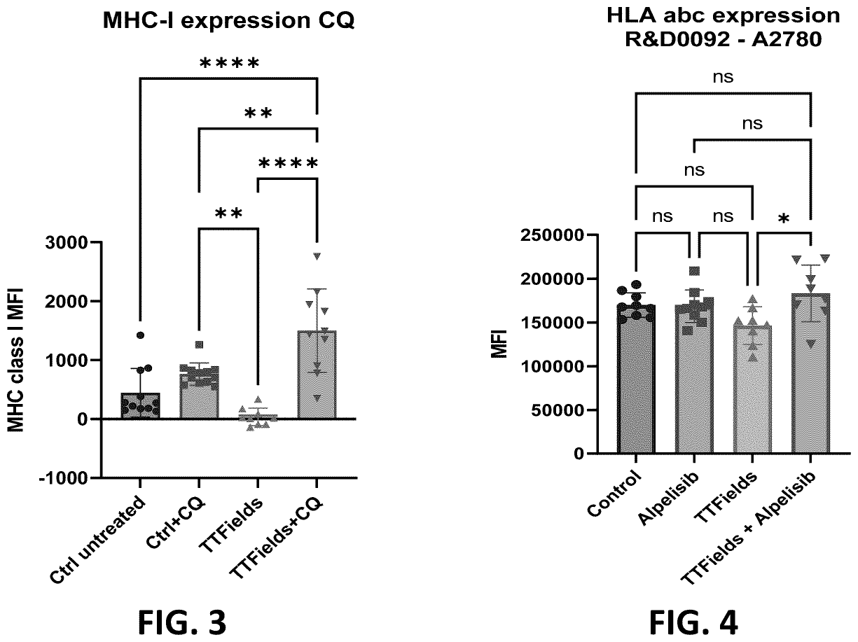

In FIG. 3, MHC Class I expression was evaluated in Panc02-luc mouse pancreatic cells (an orthotopic model of pancreatic cancer) following chloroquine (an autophagy inhibitor) treatment alone, TTFields application alone, and the combination of TTFields application with concomitant chloroquine treatment. As can be seen, MHC Class I expression was reduced in response to TTFields application; however, MHC Class I expression was increased significantly above the untreated cells when the cells were exposed to concomitant treatment with TTFields plus treatment with an autophagy inhibitor.

In FIG. 4, MHC Class I expression was evaluated in A2780 cells (an ovarian cancer cell line) following treatment with alpelisib (PI3K inhibitor) alone, TTFields application alone, and the combination of TTFields application with concomitant alpelisib treatment. As can be seen, MHC Class I expression was reduced in response to TTFields application; however, MHC Class I expression in the cells exposed to concomitant treatment with TTFields plus PI3K inhibitor was greater than in the untreated control group as well as in the single treatment groups.

### Example 3

This Example is directed to the use of TTFields in combination with composition(s) containing at least one immune checkpoint inhibitor and at least one MHC Class I expression activator for the purpose of cancer therapies. The concurrent therapy of TTFields with the composition(s) provides a synergistic effect over either treatment alone.

Human subjects are treated with TTFields by application of an OPTUNE® device (Novocure Limited, St. Helier, Jersey) to the skin of the subject, with placement of the pair of arrays left and right (LR) of the tumor and/or anterior and posterior (AP) to the tumor. Each subject is then chronically treated with TTFields at 150-200 kHz; the device is worn at least about 80% of the time, with minor breaks in between sessions and slight adjustments to the placements of the arrays, to allow the cells and skin to recover.

Two weeks after application of the TTFields has commenced, approximately 1-10 mg/kg of at least one immune checkpoint inhibitor and approximately 1-10 mg/kg of at least one MHC Class I expression activator are each orally administered or injected in the human subjects about 1-3 times a week for 6-8 weeks.

Following the concurrent therapy treatment, the effects of said concurrent therapy on the tumor(s) is evaluated.

### Non-Limiting Illustrative Embodiments of the Inventive Concept(s)

Illustrative embodiment 1. A method of reducing viability of cancer cells, the method comprising the steps of: (1) applying an alternating electric field to the cancer cells for a period of time; and (2) administering at least one composition to the cancer cells, wherein the at least one composition increases MHC Class I expression in the cancer cells. The method may be an in vitro method or an in vivo method.

Illustrative embodiment 1A. The method of illustrative embodiment 1, wherein the method is an in vitro method.

Illustrative embodiment 1B. The method of illustrative embodiment 1, wherein the method is an in vivo method.

Illustrative embodiment 2. A method of treating cancer in a subject, the method comprising the steps of: (1) applying an alternating electric field to a target region of the subject for a period of time; and (2) administering at least one composition to the subject, wherein the at least one composition increases MHC Class I expression in the cancer cells.

Illustrative embodiment 3. A method of reducing a volume of a tumor and/or preventing an increase of volume of the tumor, wherein the tumor is present in a body of a living subject and includes a plurality of cancer cells, the method comprising the steps of: (1) applying an alternating electric field to a target region of the subject for a period of time; and (2) administering at least one composition to the subject, wherein the at least one composition increases MHC Class I expression in the cancer cells.

Illustrative embodiment 4. A method, comprising the steps of: (1) applying an alternating electric field to a target region of the subject for a period of time; and (2) administering at least one composition to the subject, wherein the at least one composition increases MHC Class I expression in the cancer cells; and wherein administration of the alternating electric field increases the efficacy of the at least one composition against cancer cells in the subject when compared to the administration of the at least one composition to the subject in the absence of alternating electric field application.

Illustrative embodiment 5. The method of any of illustrative embodiments 1-4, wherein the at least one composition comprises at least one immune checkpoint inhibitor and at least one compound that increases MHC Class I expression in the cancer cells.

Illustrative embodiment 6. A method of reducing viability of cancer cells, the method comprising the steps of: (1) applying an alternating electric field to the cancer cells for a period of time; (2) administering at least one first composition to the cancer cells, wherein the at least one first composition comprises at least one immune checkpoint inhibitor; and (3) administering at least one second composition to the cancer cells, wherein the at least one second composition comprises at least one compound that increases MHC Class I expression in the cancer cells.

Illustrative embodiment 6A. The method of illustrative embodiment 6, wherein the method is an in vitro method.

Illustrative embodiment 6B. The method of illustrative embodiment 6, wherein the method is an in vivo method.

Illustrative embodiment 7. A method of treating cancer in a subject, the method comprising the steps of: (1) applying an alternating electric field to a target region of the subject for a period of time; (2) administering at least one first composition to the subject, wherein the at least one first composition comprises at least one immune checkpoint inhibitor; and (3) administering at least one second composition to the subject, wherein the at least one second composition comprises at least one compound that increases MHC Class I expression in cancer cells in the subject.

Illustrative embodiment 8. A method of reducing a volume of a tumor and/or preventing an increase of volume of the tumor, wherein the tumor is present in a body of a living subject and includes a plurality of cancer cells, the method comprising the steps of: (1) applying an alternating electric field to a target region of the subject for a period of time; (2) administering at least one first composition to the subject, wherein the at least one first composition comprises at least one immune checkpoint inhibitor; and (3) administering at least one second composition to the subject, wherein the at least one second composition comprises at least one compound that increases MHC Class I expression in cancer cells in the subject.

Illustrative embodiment 9. A method, comprising the steps of: (1) applying an alternating electric field to a target region of the subject for a period of time; (2) administering at least one first composition to the subject, wherein the at least one first composition comprises at least one immune checkpoint inhibitor; and (3) administering at least one second composition to the subject, wherein the at least one second composition increases MHC Class I expression in cancer cells in the subject; and wherein administration of the alternating electric field increases the toxicity of the at least one first composition and/or the at least one second composition against cancer cells in the subject when compared to the administration of the at least one first composition and/or the at least one second composition to the subject in the absence of alternating electric field application.

Illustrative embodiment 10. The method of any of illustrative embodiments 6-9, wherein the two administrating steps are performed simultaneously or wholly or partially sequentially.

Illustrative embodiment 11. The method of any of illustrative embodiments 1-10, wherein at least one of: the alternating electric field is applied at a frequency in a range of from about 50 kHz to about 1 MHz; the alternating electric field has a field strength of at least about 1 V/cm in at least a portion of the cancer cells/target region of the subject; the alternating electric field is induced by an applied voltage of at least 50 V RMS or at least 50 V p2p; and the period of time that the alternating electric field is applied is at least about 50% of a 24 consecutive hour time period (i.e., at least about 12 cumulative hours of a 24 hour period).

Illustrative embodiment 12. The method of any of illustrative embodiments 1-11, wherein the at least one immune checkpoint inhibitor is selected from the group consisting of a PD-1 inhibitor, a PD-L1 inhibitor, a CTLA-4 inhibitor, a TIM3 inhibitor, a TIGIT inhibitor, a LAG3 inhibitor, and combinations thereof.

Illustrative embodiment 12A. The method of any of illustrative embodiments 1-11, wherein the at least one immune checkpoint inhibitor is selected from the group consisting of a TIM3 inhibitor, a TIGIT inhibitor, a LAG3 inhibitor, and combinations thereof.

Illustrative embodiment 13. The method of illustrative embodiment 12, wherein the at least one immune checkpoint inhibitor comprises at least one PD-1 inhibitor selected from the group consisting of nivolumab, pembrolizumab, cemiplimab, spartalizumab, tislelizumab, and combinations thereof.

Illustrative embodiment 14. The method of any of illustrative embodiments 12-13, wherein the at least one immune checkpoint inhibitor comprises at least one PD-L1 inhibitor selected from the group consisting of atezolizumab, durvalumab, avelumab, BGB-A333, and combinations thereof.

Illustrative embodiment 15. The method of any of illustrative embodiments 12-14, wherein the at least one immune checkpoint inhibitor comprises at least one CTLA4 inhibitor selected from the group consisting of ipilimumab, tremelimumab, and combinations thereof.

Illustrative embodiment 16. The method of any of illustrative embodiments 12-15, wherein the at least one immune checkpoint inhibitor comprises at least one TIM3 inhibitor comprising sabatolimab.

Illustrative embodiment 17. The method of any of illustrative embodiments 12-16, wherein the at least one immune checkpoint inhibitor comprises at least one TIGIT inhibitor selected from the group consisting of vibostolimab, tiragolumab, and combinations thereof.

Illustrative embodiment 18. The method of any of illustrative embodiments 12-17, wherein the at least one immune checkpoint inhibitor comprises at least one LAG3 inhibitor selected from the group consisting of relatlimab, ieramilimab, eftilagimod alpha (soluble LAG-3 protein), and combinations thereof.

Illustrative embodiment 19. The method of any of illustrative embodiments 1-18, wherein the at least one immune checkpoint inhibitor is selected from the group consisting of nivolumab, pembrolizumab, cemiplimab, spartalizumab, tislelizumab, atezolizumab, durvalumab, avelumab, BGB-A333, ipilimumab, tremelimumab, sabatolimab, vibostolimab, tiragolumab, relatlimab, ieramilimab, eftilagimod alpha (soluble LAG-3 protein), and combinations thereof.

Illustrative embodiment 19A. The method of any of illustrative embodiments 1-18, wherein the at least one immune checkpoint inhibitor is selected from the group consisting of cemiplimab, spartalizumab, tislelizumab, BGB-A333, tremelimumab, sabatolimab, vibostolimab, tiragolumab, relatlimab, ieramilimab, eftilagimod alpha (soluble LAG-3 protein), and combinations thereof.

Illustrative embodiment 20. The method of any of illustrative embodiments 1-19, wherein the at least one compound that increases MHC Class I expression in cancer cells is selected from the group consisting of a PI3K inhibitor, an autophagy inhibitor, an HDAC inhibitor, a TRAF3 inhibitor, a BRAF inhibitor, an MEK inhibitor, topotecan, interferon-gamma, and combinations thereof.

Illustrative embodiment 20A. The method of any of illustrative embodiments 1-19, wherein the at least one compound that increases MHC Class I expression in cancer cells is selected from the group consisting of an autophagy inhibitor, an HDAC inhibitor, a TRAF3 inhibitor, a BRAF inhibitor, interferon-gamma, and combinations thereof.

Illustrative embodiment 21. The method of illustrative embodiment 20, wherein the at least one compound that increases MHC Class I expression in cancer cells comprises at least one PI3K inhibitor selected from the group consisting of alpelisib, dactolisib, PI-103, pictilisib, buparlisib, copanlisib, duvelisib, taselisib, pilaralisib, voxtalisib, nemiralisib, umbralisib, GDC-0326, SF2523, serabelisib, eganelisib, tenalisib, seletalisib, leniolisib, acalisib, parsaclisib, ME401, inavolisib, HS-173, PI-3065, GNE-317,CZC24832, GSK2636771, PF-4989216, AZD8186, AMG319, A66, AS-252424, AS-604850, CAY1505, NU75-441, CH5132799, GDC-0941, TG100-115, TG100713, CH5132799, PX-866, LY294002, XL147, ZSTK474, BKM-120, BAY80-6946, AZD8835, WX-037, KA2237, CAL-120, INCB050465, INK-1117, TGR-1202, RP6530, GDC-0032, BYL719, IPI-145, CAL-101, PIK-90,PIK-294, IC-87114, AS-605240, AZD6482, AMG511, ADZ6482, MLN1117, 3-hydroxyanthranilic acid, hispidulin, pectolinarin, cinobufagin, and combinations thereof.

Illustrative embodiment 21A. The method of illustrative embodiment 20, wherein the at least one compound that increases MHC Class I expression in cancer cells comprises at least one PI3K inhibitor selected from the group consisting of buparlisib, copanlisib, duvelisib, taselisib, pilaralisib, nemiralisib, umbralisib, GDC-0326, serabelisib, eganelisib, tenalisib, seletalisib, leniolisib, acalisib, parsaclisib, inavolisib, HS-173, PI-3065, CZC24832, PF-4989216, A66, AS-252424, AS-604850, CAY1505, NU75-441, TG100713, LY294002, PIK-90, PIK-294, IC-87114, AS-605240, and combinations thereof.

Illustrative embodiment 22. The method of illustrative embodiment 20 or 21, wherein the at least one compound that increases MHC Class I expression in cancer cells comprises at least one autophagy inhibitor selected from the group consisting of chloroquine, hydroxychloroquine, cycloheximide, bafilomycin A1, Lys05, 3-methyladenine (3-MA), LY294002, Wortmaninn, leupeptin, E64d, pepstatin A, Spautin 1, MRT 67307, MRT 68921, and combinations thereof.

Illustrative embodiment 23. The method of any of illustrative embodiments 20-22, wherein the at least one compound that increases MHC Class I expression in cancer cells comprises at least one HDAC inhibitor selected from the group consisting of apicidin, entinostat, MS-275, sodium butyrate (NaB, butanoic acid sodium salt), suberoyanilide hydroxamic acid (SAHA; vorinostat), trichostatin A (TSA), valproic acid, panobinostat, belinostat, abexinostat, dacinostat, quisinostat, mocetinostat, valproic acid sodium salt (NSC 93819, sodium valproate), CUDC-101, droxinostat, MC1568, pracinostat, divalproex sodium, givinostat, M344, romidepsin, tacedinaline, Scriptaid (GCK 1026), resminostat (RAS2410), RGFP966, RG2833, TMP269, Santacruzamate A (CAY10683), tasquinimod (ABR-215050), LMK-235, CAY10603, domatinostat, BG45, BRD72954, TMP196, tucidinostat, AR-42, citarinostat (ACY-241), GSK3117391, biphenyl-4-sulfonyl chloride, UF010, suberohydroxamic acid, NKL22, TC-H 106, SR-4370, SIS17, BRD3308, CXD101, and combinations thereof.

Illustrative embodiment 24. The method of any of illustrative embodiments 20-23, wherein the at least one compound that increases MHC Class I expression in cancer cells comprises at least one BRAF inhibitor, wherein the at least one BRAF inhibitor comprises vemurafenib.

Illustrative embodiment 25. The method of any of illustrative embodiments 20-24, wherein the at least one compound that increases MHC Class I expression in cancer cells comprises at least one MEK inhibitor, wherein the at least one MEK inhibitor comprises cobimetinib.

Illustrative embodiment 26. The method of any of illustrative embodiments 1-25, wherein the at least one compound that increases MHC Class I expression in cancer cells comprises at least one substance selected from the group consisting of chloroquine, hydroxychloroquine, alpelisib, interferon-gamma, topotecan, entinostat, trichostatin A (TSA), suberoyanilide hydroxamic acid (SAHA), sodium butyrate, apicidin, valproic acid, MS-275, vemurafenib, cobimetinib, and combinations thereof.

Illustrative embodiment 27. The method of any one of illustrative embodiments 1-26, wherein the at least one composition (or the first and/or second compositions) is/are administered before the application of the alternating electric field has begun.

Illustrative embodiment 28. The method of any one of illustrative embodiments 1-27, wherein the at least one composition (or the first and/or second compositions) is/are administered after the application of the alternating electric field has begun.

Illustrative embodiment 29. The method of illustrative embodiment 28, wherein the at least one composition (or the first and/or second compositions) is/are administered before the period of time the alternating electric field is applied has elapsed.

Illustrative embodiment 30. The method of illustrative embodiment 28 or 29, wherein the at least one composition (or the first and/or second compositions) is/are administered after the period of time has elapsed.

Illustrative embodiment 31. The method of any of illustrative embodiments 6-30, wherein at least one of the first and second compositions is administered before application of the alternating electric field has begun, and the other composition is administered after application of the alternating electric field has begun.

Illustrative embodiment 32. The method of any one of illustrative embodiments 1-31, wherein any of the steps are repeated one or more times.

Illustrative embodiment 33. The method of any one of illustrative embodiments 1-32, wherein the cancer cells/cancer/tumor is in the form of at least one solid tumor.

Illustrative embodiment 34. The method of any one of illustrative embodiments 1-33, wherein the cancers/cancer cells are selected from the group consisting of hepatocellular carcinoma/carcinoma cells, glioblastoma/glioblastoma cells, pleural mesothelioma/mesothelioma cells, differentiated thyroid cancer/cancer cells, advanced renal cell carcinoma/carcinoma cells, ovarian cancer/cancer cells, pancreatic cancer/cancer cells, lung cancer/cancer cells, breast cancer/cancer cells, and combinations thereof.

Illustrative embodiment 35. The method of any one of illustrative embodiments 1-34, wherein at least a portion of the cancer cells or at least a portion of the cancer in the subject is resistant to treatment with an immune checkpoint inhibitor alone.

Illustrative embodiment 36. The method of any one of illustrative embodiments 1-35, wherein at least a portion of the cancer cells or at least a portion of the cancer in the subject is resistant to immunotherapy.

Illustrative embodiment 37. A composition that increases MHC Class I expression in cancer cells in the subject, for use in the method of any of illustrative embodiments 1-36.

Illustrative embodiment 38. A kit for use in the method of any of illustrative embodiments 1-36, the kit comprising a composition that increases MHC Class I expression in cancer cells in the subject; and a field generating device configured to apply an alternating electric field to the cancer cells for a period of time.

Illustrative embodiment 39. A system comprising a first composition comprising at least one immune checkpoint inhibitor and a second composition that increases MHC Class I expression in cancer cells in the subject, for use in the method of any of illustrative embodiments 1-36.

Illustrative embodiment 40. A kit for use in the method of any of illustrative embodiments 1-36, the kit comprising: a first composition comprising at least one immune checkpoint inhibitor; a second composition that increases MHC Class I expression in cancer cells in the subject; and a field generating device configured to apply an alternating electric field to the cancer cells for a period of time.

While the attached disclosures describe the inventive concept(s) in conjunction with the specific experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A method of reducing viability of cancer cells, the method comprising the steps of:
(1) applying an alternating electric field to the cancer cells for a period of time;
(2) administering at least one first composition to the cancer cells, wherein the at least one first composition comprises at least one immune checkpoint inhibitor; and
(3) administering at least one second composition to the cancer cells, wherein the at least one second composition increases MHC Class I expression in the cancer cells, and wherein the at least one second composition is selected from the group consisting of an autophagy inhibitor, an HDAC inhibitor, a TRAF3 inhibitor, a BRAF inhibitor, interferon-gamma, and combinations thereof.

2. The method of claim 1, wherein at least one of:
the alternating electric field is applied at a frequency in a range of from about 50 kHz to about 1 MHz;
the alternating electric field has a field strength of at least about 1 V/cm in at least a portion of the cancer cells; and
the period of time that the alternating electric field is applied is at least about 50% of a 24 consecutive hour time period.

3. The method of claim 1, wherein step (2) is performed prior to step (3).

4. The method of claim 1, wherein step (3) is performed prior to step (2).

5. The method of claim 1, wherein the at least one immune checkpoint inhibitor of the at least one immune checkpoint inhibitor is selected from the group consisting of a PD-1 inhibitor, a PD-L1 inhibitor, a CTLA-4 inhibitor, a TIM3 inhibitor, a TIGIT inhibitor, a LAG3 inhibitor, and combinations thereof.

6. The method of claim 1, wherein steps (1) and (2) are performed wholly or partially sequentially, and wherein the at least one first composition and the at least one second composition are administered after the application of the alternating electric field has begun.

7. The method of claim 1, wherein steps (1) and (2) are repeated one or more times.

8. The method of claim 1, wherein the cancer cells are selected from the group consisting of hepatocellular carcinoma cells, glioblastoma cells, pleural mesothelioma cells, differentiated thyroid cancer cells, advanced renal cell carcinoma cells, ovarian cancer cells, pancreatic cancer cells, lung cancer cells, breast cancer cells, and combinations thereof.

9. A method of treating cancer in a subject, the method comprising the steps of:
(1) applying an alternating electric field to a target region of the subject for a period of time;
(2) administering at least one first composition to the subject, wherein the at least one first composition comprises at least one immune checkpoint inhibitor; and
(3) administering at least one second composition to the subject, wherein the at least one second composition increases MHC Class I expression in cancer cells in the subject, and wherein the at least one second composition is selected from the group consisting of an autophagy inhibitor, an HDAC inhibitor, a TRAF3 inhibitor, a BRAF inhibitor, interferon-gamma, and combinations thereof.

10. The method of claim 9, wherein at least one of:
the alternating electric field is applied at a frequency in a range of from about 50 kHz to about 1 MHz;
the alternating electric field has a field strength of at least about 1 V/cm in at least a portion of the target region of the subject; and
the period of time that the alternating electric field is applied is at least about 50% of a 24 consecutive hour time period.

11. The method of claim 9, wherein step (2) is performed prior to step (3).

12. The method of claim 9, wherein step (3) is performed prior to step (2).

13. The method of claim 9, wherein the at least one immune checkpoint inhibitor of the at least one immune checkpoint inhibitor is selected from the group consisting of a PD-1 inhibitor, a PD-L1 inhibitor, a CTLA-4 inhibitor, a TIM3 inhibitor, a TIGIT inhibitor, a LAG3 inhibitor, and combinations thereof.

14. The method of claim 9, wherein steps (1) and (2) are performed wholly or partially sequentially, and wherein the at least one first composition and the at least one second composition are administered after the application of the alternating electric field has begun.

15. The method of claim 9, wherein steps (1) and (2) are repeated one or more times.

16. The method of claim 9, wherein the cancer is selected from the group consisting of hepatocellular carcinoma, glioblastoma, pleural mesothelioma, differentiated thyroid cancer, advanced renal cell carcinoma, ovarian cancer, pancreatic cancer, lung cancer cell, breast cancer, and combinations thereof.

17. A method, comprising the steps of:
(1) applying an alternating electric field to a target region of the subject for a period of time;
(2) administering at least one first composition to the subject, wherein the at least one first composition comprises at least one immune checkpoint inhibitor; and
(3) administering at least one second composition to the subject, wherein the at least one second composition increases MHC Class I expression in cancer cells in the subject, and wherein the at least one second composition is selected from the group consisting of an autophagy inhibitor, an HDAC inhibitor, a TRAF3 inhibitor, a BRAF inhibitor, interferon-gamma, and combinations thereof; and
wherein administration of the alternating electric field increases the toxicity of the at least one first composition and/or the at least one second composition against cancer cells in the subject when compared to the administration of the at least one first composition and/or the at least one second composition to the subject in the absence of alternating electric field application.

18. The method of claim 17, wherein at least a portion of the cancer cells is resistant to treatment with an immune checkpoint inhibitor alone.
